(19) United States
(12) Patent Application Publication
(10) Pub. No.: US 2009/0025730 A1
(43) Pub. Date: Jan. 29, 2009

(54) Post decannulation stoma cover

(76) Inventor: Lloyd J. Pinel, Third Lake, IL (US)

Correspondence Address: LLOYD J. PINEL, 9 CROWS NEST CT, THIRD LAKE, IL, 60030, US

(21) Appl. No.: 12/220,088

(22) Filed: Jul. 22, 2008

Related U.S. Application Data
(60) Provisional application No. 60/961,720, filed on Jul. 24, 2007.

(51) Int. Cl. A61M 16/00

(52) U.S. Cl. 128/207.15

(57) ABSTRACT

A device and method used to cover a stoma when a patient who has had a tracheostomy tube no longer requires a tracheostomy tube and has been decannulated. The device utilizes a curved plate with an annular foam or air-filled cushion, an adhesive and a trach tie to hold the device in place over the stoma. The device effectively blocks the stoma to allow for speech, promote wound healing and prevent infection.

### CROSS-REFERENCE TO RELATED APPLICATIONS

This application claims the benefit of provisional application No. 60/961720. Filed on Jul. 24, 2007.

### STATEMENT REGARDING FEDERALLY SPONSORED RESEARCH OR DEVELOPMENT

n/a

### REFERENCE TO SEQUENCE LISTING

n/a

### BACKGROUND OF THE INVENTION

This invention relates to the field of medical care, specifically the care of a patient immediately following the planned removal of a patient's tracheostomy tube.

A tracheostomy tube is a short curved tube inserted through a surgical hole in the patient's neck and held in place with a tracheostomy tie around the patient's neck. The purpose of a tracheostomy tube is to provide the patient with a patent airway.

Patients have tracheostomy tubes placed for numerous reasons. Once the reason that necessitated the placement of a tracheostomy tube has been resolved, the tracheostomy tube is normally removed. This procedure is known as decannulation. After the patient is decannulated there remains a hole in the patient's neck which is known as a stoma. Once the patient is decannulated the stoma will close on its own in approximately fifty percent of the patients. This normally occurs in two to three weeks. Surgical closure of the stoma is required for the patients who's stomas do not close on there own.

Currently when a patient is decannulated, a pressure bandage type of arrangement is placed on the patient for the first day or so while the patient is in the hospital. Once the patient leaves the hospital, the usual method of covering the stoma is to use a Band-Aid. The patient is then instructed to cover the stoma with a finger when the patient needs to speak or cough.

This method of covering the stoma with a Band-Aid, and using a finger to occlude the stoma in order to talk, suffers from a number of disadvantages. First, if the patient is a child, or a developmentally or physically disabled adult, he or she may not be able to be taught to occlude the stoma with a finger as needed. Second, in all patients, if the stoma is not covered in a somewhat airtight fashion, air passing in and out of the stoma when the patient attempts to talk, causes difficulty with or even makes it impossible for the patient to have the ability to speak. Third, when the patient coughs without covering the stoma, air is forced out of the stoma, even normal breathing causes air to pass in and out of the stoma if only covered by a band-Aid. This air movement through the stoma reduces the chance of the stoma closing on its own without surgery. Fourth, touching the stoma with the patient's fingers to talk or cough, greatly increases the chances for infection of the patient's stoma and airway.

The devices currently available which relate to stomas, are all designed to either filter the air being inhaled or to keep the stoma open in patients who need to have their stoma open permanently.

Currently there is no device available which adequately keeps air from flowing in and out of a decannulated patient's stoma. Therefore, there is a need for such a device to be developed.

### BRIEF SUMMARY OF THE INVENTION

In view of the foregoing, it is a primary object of the present invention to provide a device which will effectively prevent air from moving into and out of the stoma of a patient who has been decannulated, with out the patient needing to manually occlude the stoma with a finger in order for the patient to have the ability to speak. This device provides a curved plate which covers the stoma and is held in place, such as with a strap or tracheostomy tie around the patient's neck. This also provides the advantage of reducing the chance of infection at the site of the patient's stoma due to germs present on the patient's fingers.

Another object of this invention is to provide a comfortable airtight seal around the patient's stoma without having pressure applied directly to the site of the stoma. This is accomplished by providing a foam or air filled annular ring which places the pressure supplied by the curved plate and the strap, to the area around the outside of the patient's stoma.

Another object of the invention is to provide a means to prevent the device from slipping away from the stoma because of patient movement. This is accomplished by providing an adhesive on the patient side of the foam or air filled annular ring to help anchor the device in place.

These and other objects of the present invention will become apparent to those skilled in the art from the drawings and the following description.

### BRIEF DESCRIPTION OF DRAWINGS

FIG. 1 is a front view of a preferred embodiment of the present invention.

FIG. 2 is a back view of a preferred embodiment of the present invention.

### DETAILED DESCRIPTION OF THE INVENTION

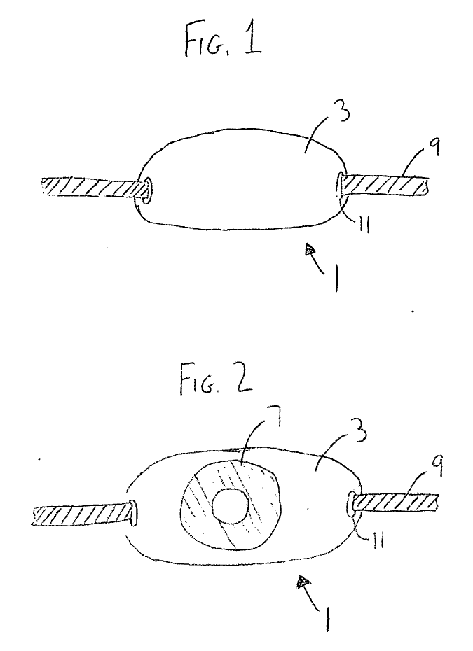

Referring to FIGS. 1 and 2, the device 1 has a plate 3 which fits over the patient's stoma. The plate 3 is preferably curved to fit the natural curvature of the patient's neck, and should be large enough to cover the stoma.

Attached to the patient's neck side of the plate 3 is a cushion 7. The cushion 7 may be adhered to the plate 3 by adhesive or any other suitable method. Additionally, the cushion 7 may be permanently or temporarily adhered to the plate 3. The cushion 7 preferably forms a circle around the stoma, so that pressure from the device 1 does not press directly against the stoma. The cushion 7 is preferably foam or an air-filled annular or donut-shaped pillow to surround the stoma. Preferably, the cushion 7 would have adhesive to adhere to the patient's neck and anchor the device 1 in place. Although the shape in FIG. 2 is shown as circular on both the outer and inner perimeter of the cushion 7, the shape can be any suitable shape for adhering to the patient's skin without contacting the stoma, with the inner and outer perimeter being any suitable combination of circular, oval, rectangular, square, rounded rectangular, rounded square, trapezoidal, or other suitable shapes.

The plate 3 can optionally be attached to a strap 9, which may be a tracheostomy tie or an elastic band. The purpose of the strap 9 is to provide additional pressure against the patient's neck and to further secure the device 1 in place. The strap 9 may be attached to the plate 3 through openings 11 in the plate 3, or by any other suitable method.

The plate 3 can be of any shape, but is preferably approximately oval in shape, so as to cover the stoma, adhere to the cushion 7, and attach to the strap 9. The shape of the plate 3 any suitable shape such as circular, oval, rectangular, square, rounded rectangular, rounded square, trapezoidal, or other suitable shape.

The plate 3 is preferably manufactured using plastic to reduce costs but can be made of metal, composite or any other suitable material.

The device 1 will help to keep air from flowing into and out of the stoma, thereby facilitating the patient's speech and increasing the chance of the stoma closing on its own, which could reduce the need for surgery to close the stoma. The device 1 is preferably disposable and should be changed every 24 hours to help prevent infection.

In accordance with the provisions of the patent statutes, the present invention has been described in what is considered to represent its preferred embodiment. However, it should be noted that the invention can be practiced otherwise than as specifically illustrated and described without departing from its spirit and scope.

What I claim is:

1. A device for providing a cover to prevent air from moving in and out of a stoma on a patient's neck, comprising:
a plate, disposed to cover the stoma and containing slots; and
a strap threaded through the slots and around the neck, to hold said plate over the stoma.

2. A device as in claim 1, wherein said plate is curved to conform to the shape of the neck.

3. A device as in claim 1, further comprising an annular-shaped cushion attached to said plate and positioned to form a seal around the stoma.

4. A device as in claim 3, further comprising an adhesive on said cushion to assist in anchoring the device to the neck.

5. A device as in claim 3, wherein the annular-shaped cushion is foam.

6. A device as in claim 3, wherein the annular-shaped cushion is filled with air.
